(19) United States
(12) Patent Application Publication
(10) Pub. No.: US 2011/0054238 A1
(43) Pub. Date: Mar. 3, 2011

(54) Titanium ionic/Magnetic Bracelet

(76) Inventor: Katsumi Komatsu, Chuo-Ku (JP)

(21) Appl. No.: 12/547,291

(22) Filed: Aug. 25, 2009

(51) Int. Cl. A61N 2/08

(52) U.S. Cl. 600/15

(57) ABSTRACT

A titanium alloy bracelet with embedded magnets and a source of negative ions is presented. The embedded magnets are permanent and arranged in a patented alternation north-south orientation to confer improved magnetic field strength. The titanium bracelet helps to disperse the negative ions produced by the ion source.

### FIELD OF THE INVENTION

This invention relates to health improvement devices and non-drug treatment of health problems.

### BACKGROUND OF THE INVENTION

Ionic treatment of human health problems is an old technique. Minus Ions are generally abundant in nature through plants, waterfalls, rain storms and forests: all of which offer a natural good feeling. These Ions unfortunately are depleted in urban areas as a result of modern technology.

The human body absorbs positive ions from electronic equipment, cell phones, electrical wiring, and machinery encountered in everyday modern life. Physical stress and exposure to Ultraviolet (UV) Rays also elevates the positive ion levels within the body. Research shows that excessive Positive Ion levels are associated with fatigue, lack of concentration, muscle and joint aches, and feeling of nausea.

The present invention's Minus-ion properties counteract the Positive Ions created by modern technology, daily stress, and physical activity.

From the times of the ancient Egyptians, Eastern Culture has embraced magnets for their therapeutic powers. Magnetic fields exist in all areas of life. The earth is in essence one large magnet, surrounded by a large magnetic field. The earth's magnetic field protects us from radiation.

The human body also responds to magnetic fields, restoring healthy blood flow to the arteries and veins. Magnets tend to soften human tissue around blood vessel walls, thereby increasing the elasticity of the blood vessel and lowering blood pressure.

The present invention possesses ionic and magnetic properties and can be worn as a bracelet, and thereby permitting direct contact with the human body. The titanium metal of the bracelet itself has important effects in that it helps the human brain release alpha waves.

### SUMMARY OF THE INVENTION

The present invention consists of a titanium metal bracelet with a stone in the center of the bracelet and two magnets, one at each open end of the bracelet.

The Ions in the current invention are released by the stone in measurable and in significant amounts. The design of the present invention produces twenty to fifty times more Minus Ions than state-of-the-art Ion bracelets.

The design of the present invention permits it to be washed, and it will still maintain its minus-ion producing properties when wet, and can be worn while swimming or surfing.

The cells in the body create electrical charges and magnetic forces. Human cells require magnetic fields for health and regeneration. Modern technology and lifestyles shield us from the earth's natural magnetic fields. The present invention creates a boost in the body's magnetic field, embracing its natural active aura.

The present invention uses medical grade magnets in a unique Alternating North-South Polarity Orientation (ANSPO) to maximize magnetic field flow, to increase the effective area of the magnetic field, and penetrate deeper into tissue fibers and muscle. This technology is taught by U.S. Pat. No. 6,913,663 to Komatsu.

Prior art magnetic devices on the market utilize two-dimensional magnets which are inexpensive to produce and have minimal magnetic field penetration. Other multi-magnet devices orient “Like” poles in the same direction. This orientation is less effective due to the fact that “like” poles repel, creating a dead zone with no magnetic field between the magnets.

### BRIEF DESCRIPTION OF THE FIGURES

FIG. 1. View of the invention

FIG. 2. Alternating, north-south polarity orientation

FIG. 3. Magnet placement on invention

FIG. 4. Ionic material capsule placement

### DETAILED SPECIFICATION

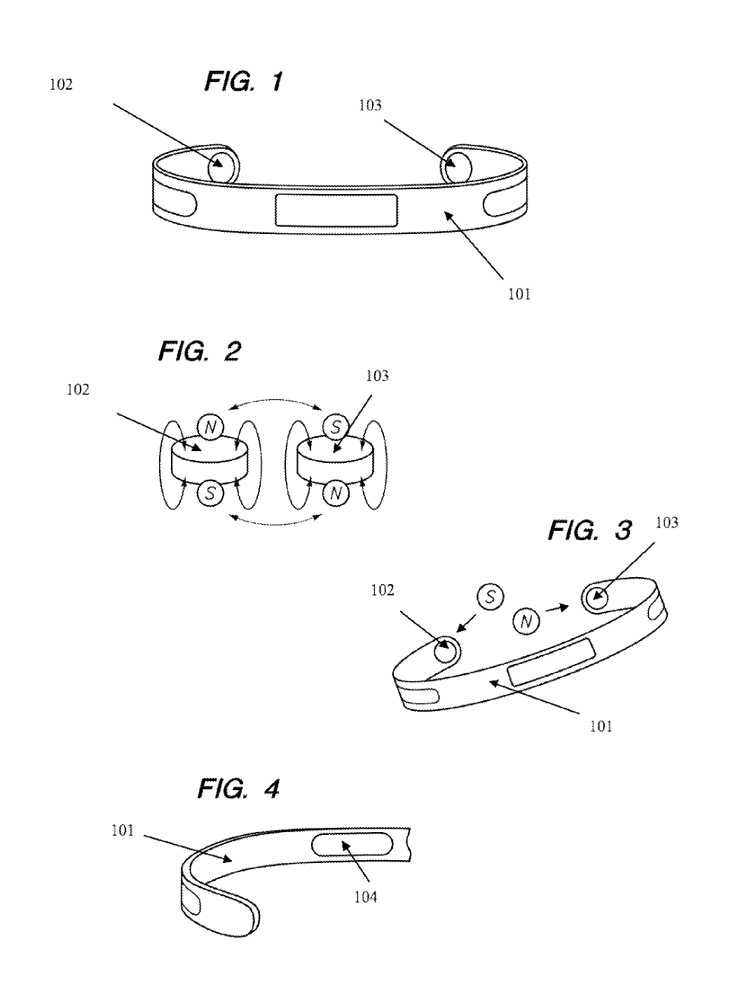

The invention 100 is comprised of a length of titanium metal alloy 101 with two permanent magnets 102, 103 at each end with a stone 104 that emits negative ions at the midpoint of the length. The preferred embodiment of the invention is a solid band of titanium metal alloy 101 in the shape of a bracelet, as in FIG. 1.

The two permanent magnets 102, 103 are arranged at the ends of the arms of the bracelet such that they attain an alternating, north-south polarity orientation, as described in U.S. Pat. No. 6,913,663 and illustrated in FIG. 2 and FIG. 3. The magnetic strength of these permanent magnets 102,103 is approximately 1,000 Gauss. The specific, patented orientation of the two permanent magnets in proximity to each other results in an increased flow of magnetic flux lines between the magnets, which is beneficial to the wearer of the invention.

The stone 104 is selected from a set of ion-producing materials, including but not limited to tourmaline. The stone 104 is set at the midpoint of the invention, as shown in FIG. 4, to maximize the reach to the wearer of the negative ions it emits. the combination of a negative ion source such as the stone 104 with a titanium bracelet 101 boosts the ion power of the invention up to 100 times of the strength of a ionized metal bracelet by itself.

The combination of negative ion production and alternating, north-south pole oriented permanent magnets 102,103 confers an increased benefit to the wearer that use of either alone would not, including increasing blood flow and softening of the walls of blood vessels to increase flexibility and decrease blood pressure.

The technology to create the apparatus above and the apparatus listed as the preferred embodiment of this invention, and other methods, are possible and are within the contemplation of this patent.

What is claimed is:

1. a titanium alloy bracelet, comprised of a length of titanium metal alloy, two permanent magnets, and a capsule,
the length of titanium metal alloy in an open circular shape, the capsule embedded in the length of titanium metal alloy at the midpoint of its length, the permanent magnets placed at the ends of the length,
the capsule comprised of material that emit negative ions,
the permanent magnets comprised of naturally magnetic material,
the permanent magnets embedded at the ends of the length of titanium in an alternating, north-south polarity orientation.

2. The titanium alloy bracelet as in claim 1, where the length of titanium metal alloy is a solid band in the shape of a bracelet.

3. The titanium alloy bracelet as in claim 1, where the material that emits negative ions is tourmaline.
